(19) United States
(12) Patent Application Publication
(10) Pub. No.: US 2023/0109850 A1
(43) Pub. Date: Apr. 13, 2023

(54) System for and Method of Direct Marketing Takeout Menus for Point of Sale Systems

(71) Applicant: Quyen T. Kiet, Huntington Beach, CA (US)

(72) Inventor: Quyen T. Kiet, Huntington Beach, CA (US)

(21) Appl. No.: 17/652,966

(22) Filed: Mar. 1, 2022

Related U.S. Application Data
(60) Provisional application No. 63/262,279, filed on Oct. 8, 2021.

(51) Int. Cl. G01N 33/02; A23L 35/00; G06F 16/27

(52) U.S. Cl. CPC G01N 33/02; A23L 35/00; G06F 16/27; A23V 2002/00

(57) ABSTRACT

A system enables a consumer to place an order for a food item by entering characteristics and not necessary by its commercial name. The system prompts the consumer to enter the information about the desired food item and selects food items from a food database according to the characteristics entered. The selected food items are presented to the consumer for confirmation and the confirmed food item is sent to a selected restaurant for fulfillment.

### CROSS-REFERENCE TO RELATED APPLICATIONS

This application claims is a non-provisional of U.S. Provisional Patent Application 63/262,279, filed on Oct. 8, 2021, for “DINER MARKETING AND POINT-OF-SALE” (DMAP) IS A SYSTEM FOR AND METHOD OF DIRECT MARKETING TAKEOUT MENUS FOR POINT OF SALE SYSTEMS, the specification of which is incorporated here by its entirety by this reference.

### TECHNICAL FIELD

This invention relates to restaurant services, more specifically, relates to automated dish identification.

### BACKGROUND OF THE INVENTION

The advent of the Internet facilitates much restaurant business, the Internet made possible for consumers at home to browse and to order food from the comfort of their homes. Because of the Internet, now the consumers have access to many food ordering websites; however, having access to these food ordering websites does not mean that a consumer can easily place an order for his desired product because he may not know the exact name of the dish he wants or he may not be confident in the taste of the dishes displayed on the menu.

Therefore, there is a need for a system and method that facilitates ease for consumers to place orders for food items and it is to this system to which the application is primarily directed.

### SUMMARY OF THE INVENTION

In one embodiment, disclosed is a method for assembling a dish database which comprises receiving a description for a food item, performing chemical analysis on a sample for the food item, receiving a result from the chemical analysis; and populating the dish database with the result and the description.

In another embodiment, disclosed is a method for receiving an order from a consumer. The method comprises displaying a menu to the consumer, receiving an input from the consumer, if the input is an audio input, performing a voice recognition on the audio input to obtain a name for a food item, if the input is an image input, performing an image recognition on the image input to obtain a name for the food item, if the input is a list of ingredients, performing a search on an ingredient database for the list of ingredients to obtain a name for the food item, checking the name against a dish database to see if the food item is available, if the food item is not available, generate a palate index for the food item, if the food item is not available, selecting a substitute food item based on the palate index, and if the food item is not available, presenting the substitute food item to the consumer.

In yet another embodiment, disclosed is an apparatus for receiving an order from a consumer. The apparatus comprises a display device for displaying a menu to a consumer, a data communication device for interfacing with remote consumers and restaurants, an IO controller for controlling input/output for the apparatus, a storage unit for storing dish information, customer information, restaurant information, and program code, a dish analyzer for analyzing content of food item, and a controller for executing the computer programs. When the controller executes the computer programs, the controller causes the apparatus to display a menu to the consumer, receive an input from the consumer, if the input is an audio input, perform a voice recognition on the audio input to obtain a name for a food item, if the input is an image input, perform an image recognition on the image input to obtain a name for the food item, if the input is a list of ingredients, perform a search on an ingredient database for the list of ingredients to obtain a name for the food item, check the name against a dish database to see if the food item is available, if the food item is not available, generate a palate index for the food item, if the food item is not available, select a substitute food item based on the palate index, and if the food item is not available, present the substitute food item to the consumer.

Other advantages and features of the present invention will become apparent after review of the hereinafter set forth Brief Description of the Drawings, Detailed Description of the Invention, and the Claims.

### DESCRIPTION OF THE DRAWINGS

Features and advantages of embodiments of the invention will become apparent as the following detailed description proceeds, and upon reference to the drawings, where like numerals depict like elements, and in which:

FIG. 1 depicts a use scenario 100 of the present invention;

FIG. 2 is a schematic 200 for menu construction;

FIG. 3 is a process 300 for identifying a specific dish;

FIG. 4 is a process 400 for taking order from a user;

FIG. 5 is a process 500 for selecting a restaurant;

FIG. 6 is a schematic 600 of a server; and

FIG. 7 is process 700 for user input.

### DETAILED DESCRIPTION OF THE INVENTION

In the restaurant space the preference for takeout (offering diners the option of taking their dishes out “to go” rather than dining-in) over dining-in at the restaurant premises has steadily increased. With social distancing and lockdowns (health measures in response to pandemics) mandated in many states in 2020 and 2021, restaurants had no choice but to turn to takeout and delivery options in order to serve diners. To facilitate and assist restaurants which offer takeout and delivery options, the subject invention is a system and method that connects restaurants to prospective diners by enabling search and discovery of specific restaurant dishes, payment for said dishes and direct marketing to diners. Thus, this invention benefits diners as it makes it easy to find specific dishes nearby for takeout or delivery and enriches restaurants as it not only attracts new diners but enables direct sales and optimizes marketing of specific dishes. With these value propositions in mind, this invention is referred to henceforth as the Diner Marketing and Point of Sale System and abbreviated as “DMAP.”

Current solutions popularly available online are centered around restaurant discovery and review rather than individual dishes for discovery, payment and promotion. DMAP is designed to be dish specific discovery and marketing, and more uniquely, integrates payment processing of individual dishes so that it is an end-to-end solution that publishes the restaurant's menu for discovery, payment, marketing and business analysis at the menu item level (per dish).

The objectives of DMAP are:
1. For Diners:
   a) Search for a specific dish for takeout nearby;
   b) Discovery of new dishes available for takeout;
   c) Payment processing for selected dishes;
   d) Discovery of similar dishes for payment and takeout at an alternate restaurant nearby when the selected dish is not currently available (if the selected restaurant is not yet opened).
2. For Restaurants:
   a) Marketing of specific dishes for discovery and purchase by prospective diners;
   b) Discovery by new diners searching something else but discovering the restaurant's related dishes for the first time;
   c) Discovery by new diners searching for a specific dish which the restaurant offers for takeout;
   d) Direct payment for takeout orders of dishes found on DMAP (via DMAP's integrated point of sale system or a third-party point of sale system);
   e) Marketing of specific dishes directly to past diners who have previously paid the restaurant for takeout on DMAP;
   f) Marketing of specific dishes to unknown diners who have previously purchased similar dishes on DMAP at a different restaurant;
   g) Tracking and analysis of diner preferences relative to the restaurant's offered menu;
   h) Market analysis per dish;
   i) Menu design based on diner preferences and habits;
   j) Quick discounts (price reductions and promotion) of dishes to nearby diners for a fixed duration of time;
   k) Inventory management of perishable ingredients.

Definitions of terms used herein are as follows:

“Diner”: Restaurant customer(s) or prospective customers open to buying dishes from a restaurant.

“Dish”: Any item on a restaurant's menu that can be purchased for takeout and delivery by the diner. A dish can be described as a specific item on the menu aka “menu item” which the restaurant intends to be sold to the diner.

“Menu”: The collective list of dishes offered by a restaurant for purchase and consumption.

DMAP consists of six key functions:
1. Publication. The restaurant's menu is made public and discoverable via DMAP's publication function. This function is available on multiple platforms unlimited in scope and generally embodied as an interactive layer between the public and the content such as a website on the internet, mobile application (“app”) on a smart phone or application (“widget”) on a social media site. DMAP connects the restaurant's menu information to the public layer as follows:
a) Menu Entry. The restaurant publishes its menu for public discovery as described above by entering the necessary information (such as name, price, ingredients, photo, video, etc.) into the DMAP software client on the Restaurant's computer.
b) Distribution to Discovery Tools. DMAP takes the information entered and serves it to all connected public facing platforms (websites, smart phone apps, widgets, etc.) via these public facing discovery tools optimized for menu search.
c) Third Party Access. A marketing company or third party may enter all the necessary menu information on behalf of the restaurant.
d) Indexed for Search. All menu information entered is fully indexed to enable ease of search and discovery as described below.
e) Language Conversion. Dishes spelled out in a foreign language are indexed and correlated with English nomenclature for discovery purposes. For example, if the restaurant enters the dish as “pho”, DMAP has a language conversion feature that allows diners searching for “noodle soup” to find “pho” in the results because “pho” is a style of Vietnamese noodle soup.
2. Discovery Tool. DMAP has public facing channels to enable the expeditious search and discovery of specific menu items by diners. This discovery tool is the interface between diners and a restaurant's published content relating to information about their menu. Diners can access DMAP's Discovery Tool and discover restaurant dishes via DMAP's smartphone app, website, widgets, etc.
   a) Search. To find a specific dish, diners enter the name of the dish and DMAP identifies dishes which best match the search then filtered and sorted based on proximity to the diner, price preference of the diner, and availability of the restaurant (open or closed).
   b) Best Guess. If the specific dish is not available but similar dishes are available nearby, then these dishes will also be shown for selection and purchase.
   c) Deals. Similar dishes available nearby that are designated as restaurant promotions (described above under Promotions) are highlighted with the discount.
   d) Language Conversion. Some dishes may have uniquely foreign names that do not match the spelling as entered by the diner but is actually the dish for which the diner is searching. As described above in the Language Conversion feature for Publication, the Vietnamese dish often found in menus as “pho” can be discovered by diners unfamiliar with the dish but are looking for noodle soup. When the diner types in “noodle soup”, foreign dishes that are also described as noodle soup will be included in the result such as “pho” and “ramen” style noodle soups.
   e) Selection. From the search results, if diners see a dish they want to select for takeout, they can select it and the Discovery Tool leads them to either DMAP's integrated point of sale system (Payment described separately below) or to the restaurant's third-party POS system.
   f) Discovery. Diners can discover new dishes thanks to suggestions of different or alternate options as part of DMAP's search results based on the content of the dish and selection history of other Diners. For example, if the diner is searching for “brisket noodle soup” but none or few such dishes are available nearby, then DMAP would suggest “flank noodle soup” because DMAP's Discovery function recognizes that both dishes share style and content: soup, noodle and beef. Additionally, this alternate suggestion is further filtered for priority if other Diners also select “flank noodle soup” when searching for either “brisket noodle soup” or “beef noodle soup” or simply “noodle soup”. And when combined with the Language Conversion feature, the relevance and usefulness of discovery improves over time as more information of diner search selections are archived and analyzed over time. For example, when searching for “brisket noodle soup”, the Discovery function will offer “pho flank” in addition to “pho brisket” because DMAP's Language Conversion feature recognizes that “pho” also means noodle soup.
3. Payment (POS). DMAP has an integrated point of sale (“POS”) system that restaurants may elect to enable such that when diners select a dish to purchase for takeout, the payment can be directly sent to the restaurant via DMAP's POS system. Alternatively, if the restaurant prefers, DMAP may direct the Diner to a third-party POS system for completion of the sale.
   a) Using DMAP's integrated POS system allows the restaurant to integrate its POS pricing to the Promotion function because when the restaurant changes the price of a dish on DMAP's POS, it can be designated as a “promotion” (described below) to attract more diners, this new price is re-published on all of DMAP's public facing discovery tools and highlighted as a “promotion” accordingly to draw the diners' attention to the pricing discount.
4. Promotion. The Promotion function allows restaurants to draw interested diners to specific dishes by timely lowering the prices of said dishes and having such discounts be highlighted as a special deal or discount. The Promotion is unique because it offers the following benefits:
   a) “Interested” Diners. Based on the diners' history of purchases, diners are considered “interested” if they have previously purchased, searched or selected this dish at any time while using DMAP.
   b) Restaurants are able to identify which dishes are most popular to nearby diners and at what time of the day. For example if “egg sandwich” is the most popular dish being searched by interested diners on DMAP nearby at 8:00 AM on Fridays, then DMAP will automatically place promotions for egg sandwiches on Fridays around 8:00 AM—the time when interested diners are most likely to discover and buy for this dish.
5. Menu Analytics. DMAP analyzes the search and purchase history of interested diners within any defined distance from the restaurant to provide the restaurant with insight as to which dishes are most commonly searched, selected and purchased on DMAP.
   a) DMAP analyze a menu to determine which dishes are most popular by showing (1) when they are most popular (2) where they are most popular and (3) the price range of such dishes.
   b) DMAP can further create market analysis reports and business analytics for the restaurant to make better financial decisions as to when to buy ingredients during the month, season and year.
   c) If the restaurant enters the requisite inventory information (purchase date and expiration date of perishable ingredients for every dish) DMAP can assist with inventory management by identifying which dishes have perishable ingredients nearing their expiration date and make recommendations for such dishes to be on Promotion for quicker liquidation of perishable ingredients nearing the expiration date.

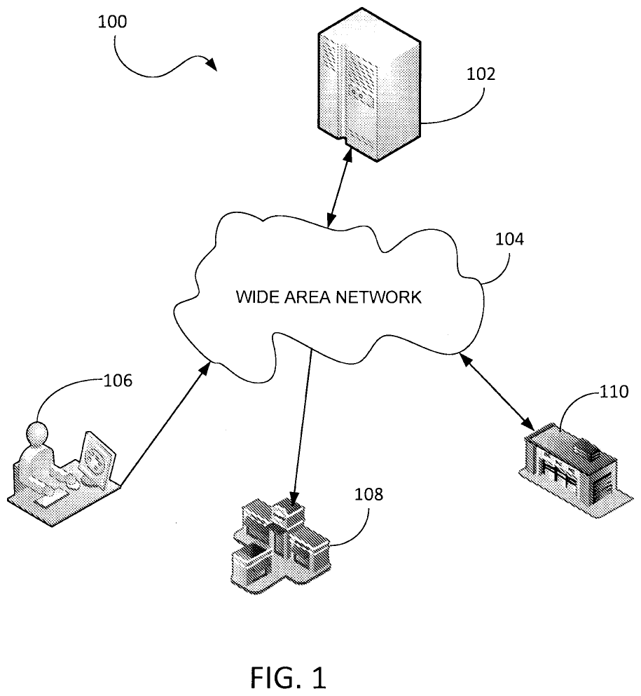

The system developed according to the DMAP supports interaction through a wide area network 104 (the Internet) between a consumer 106 and a restaurant 108. FIG. 1 illustrates a scenario 100 of such interaction. The dishes provided by restaurants are cataloged initially by a publisher 110. The publisher 110 lists different dishes commonly offered by the restaurants 108 according to different factors. This process will be further described later. The list of dishes is stored in a server 102 that is accessible to the consumers 106 through the Internet 104. When a consumer 106 wants to order a dish for take-out or delivery, the consumer 106 access a portal on the internet 104 and makes a selection. The selection is received by the server 102, recorded in a database, and transmitted to a selected restaurant 108 for fulfilment. The selected restaurant 108 prepares the selected dish according to the order received from the server 102.

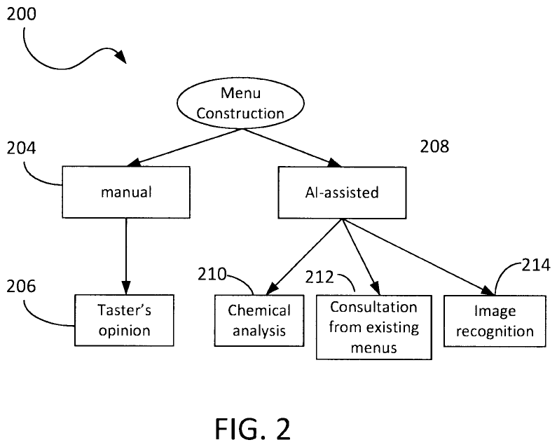

The publisher 110 assembles and maintains the menu for use by the consumers 106. The menu can be assembled in many ways but basically in two categories, manual 204 or Al-assist (Artificial-Intelligence) 208 as shown by schematic 200 of FIG. 2. The manual way 204 consists of humans entering dish names into the database and also entering the characteristics and the taste of the dishes into the database. The characteristics and the taste base based on the personal opinion 206 of the taster. If the taster has the actual dish, he may use chemical analysis 210 to determine whether the dish is spicy, salty, sweet, etc. The taster may also consult existing menus 212 to see how the dish in question is described. If the taster has an image of a dish, he may use image recognition program 214 to identify the dish and once he identifies the dish, he may use other methods to derive other characteristics of the dish.

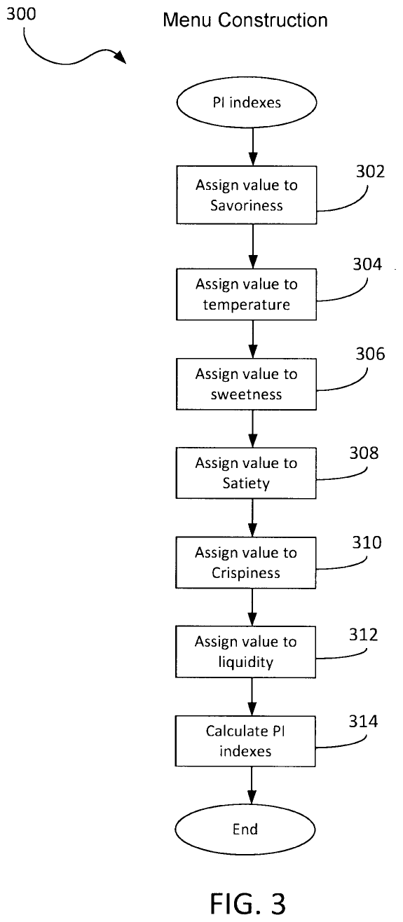

The database of the dishes can also be populated by the Al-assist method 208. The Al-assist method relies, at least partially, use of a machine (computer) to assess the dishes and to derive proper information related to the dishes. The pertinent information related to each dish may include flavor, temperature, sweetness, crispiness, etc. FIG. 3 illustrates an exemplary process 300 to derive a palate index (PI). The palate index for each dish is calculated based on different factors. The PI process starts with assigning a value to savoriness, step 302, to temperature, step 304, to level of sweetness, step 306, to satiety, step 308, to crispiness, step 310, and to liquidity, step 312. After all these factors are calculated, the PI can be easily derived, step 314. The definitions for each of these factors are:
1. Savoriness: how salty or spicy a dish may taste
2. Temperature: how hot a dish should be served
3. Sweetness: how sweet a dish may taste
4. Satiety: how quickly the dish makes the diner feel full; for example, a burrito filled with rice, beans, meat and cheese has high satiety vs a bowl of chicken noodle soup
5. Crispiness: how much crunchiness or crispiness in the first bite of the dish (apples and potato chips have high crispiness while porridge has none)
6. Liquidity: how much fluid is in a dish; maximum liquidity would be a broth while minimum liquidity would be a potato chip

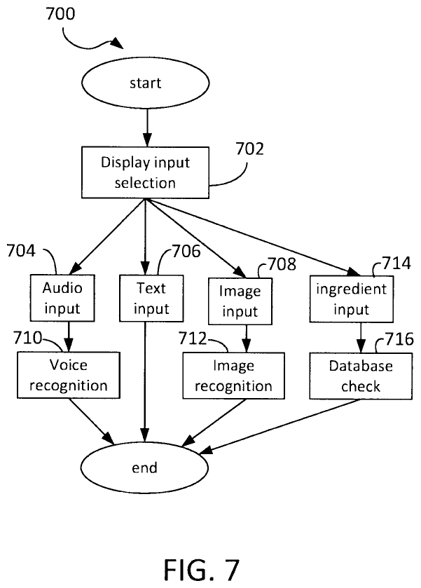

FIG. 7 is a user input process 700. When a consumer is ready to order a dish, the consumer may access the DMAP system through the Internet or direct dial and a menu is displayed to the user, step 702. If the consumer may input the information about the desired dish by entering the common name of the dish, step 706, for example, Kung Pao chicken. If the consumer enters a not commonly used name, the DMAP system will check a database for dishes that potentially match what the consumer is looking for.

However, if he does not know the common name for the desired dish, then the special features of the DMAP system will become handy. He may enter a picture of the desire dish, step 708, and the DMAP system will use an image recognition system, step 712, to identify the most likely candidate for the dish and then prompt the consumer to confirm. If the consumer is using a handset and he may speak the dish name, step 704, and a voice recognition software will be employed to recognize the dish name, step 710.

The consumer may also enter the ingredients of a dish, step 714. For example, the consumer may desire to have a dish that include broccoli or cauliflower. The DMAP system will search the database, step 716, for dish that includes broccoli or cauliflower. The voice recognition software 710 and the image recognition system 712 are preferably resident on the DMAP system.

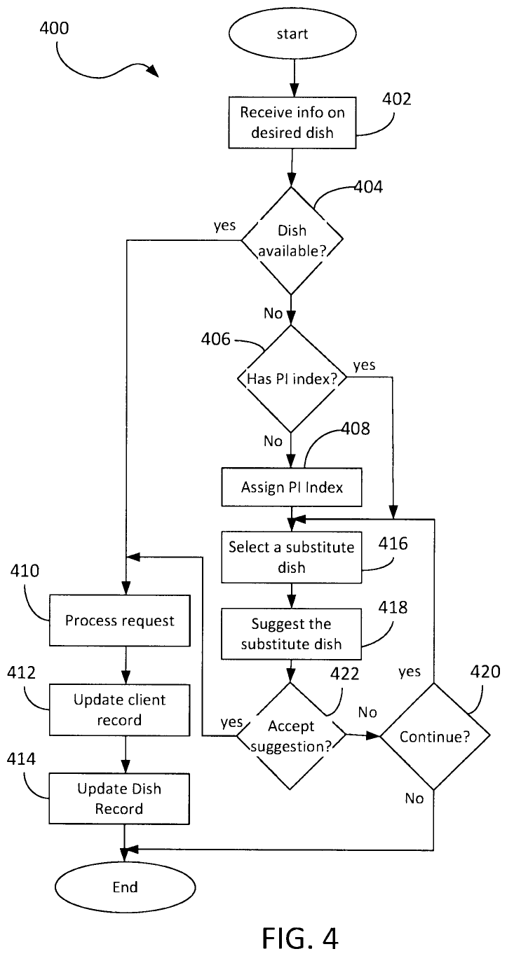

FIG. 4 is a process 400 for handling order from a consumer. After the DMAP system received a desired dish selection by either of the methods described above, step 402, the DMAP system checks whether the dish is available, step 404, in the database. The consumer may also enter the ingredients of a desired dish, such as beef, onion, and potato. The DMAP system will search the database for the suitable dish according to the information received. The DMAP system may calculate a PI index for the information received and then select a dish with a similar or identical PI index. If the dish is available, the consumer's request is processed, step 410, by sending it to a restaurant for fulfillment. The consumer's record will also be updated, step 412, so the consumer's preference is tracked. The dish database is also updated, step 414, to reflect the popularity of the dish.

If the dish is not available in the database, the DMAP system checks whether a PI index has been assigned, step 406. If there is no PI index associated with the dish, the DMAP system assigns a PI index, step 408, to the dish as described previously. After the dish has a PI index, it can be check if there is a substitute dish with a similar PI index, step 416 and the DMAP system will present the substitute dish as an alternative to the consumer, step 418. If the consumer accepts the suggestion, step 422, the substitute is sent for processing, step 410. If the consumer rejects the suggestion but it is open for another suggestion, step 420, the DMAP system will proceed to select a different substitute dish, 416.

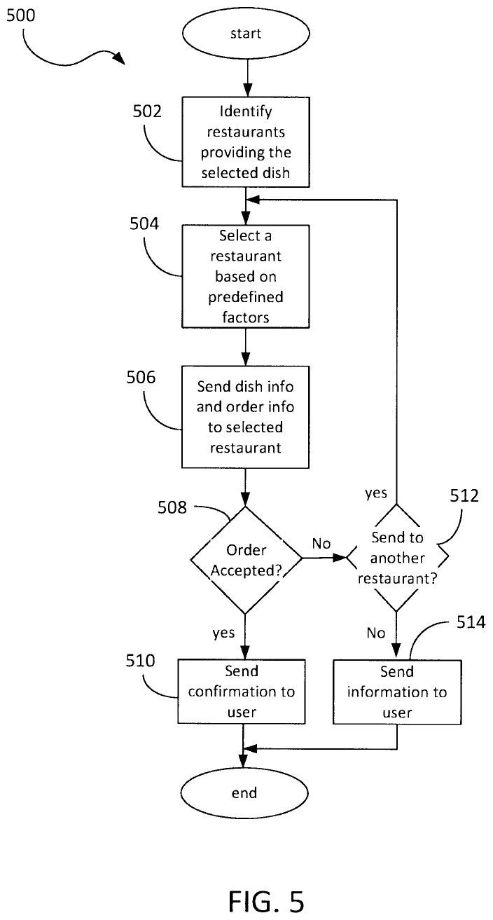

After the dish has been identified and accepted by the consumer, the dish is sent to a restaurant for fulfillment. FIG. 5 is a process 500 for selecting a restaurant. The DMAP system identifies restaurants that can provide the selected dish, step 502 and then further select a restaurant based on predefined factors, step 504. The predefined factors can be set by the consumer, for example, based on geographical information or promotional information input by the consumer. The predefined factors may also be set according to the agreement between the restaurants and the publisher, the operator of the DMAP system. The DMAP system will send the dish information to the selected restaurant, step 506. If the restaurant accepts the order, step 508, a confirmation will be sent to the consumer, step 501. If the select restaurant declines to fulfill the order, the dish information may be sent to another restaurant, step 512. If there is no available restaurant to take the order, this information will be sent to the consumer that the order cannot be fulfilled, step 514.

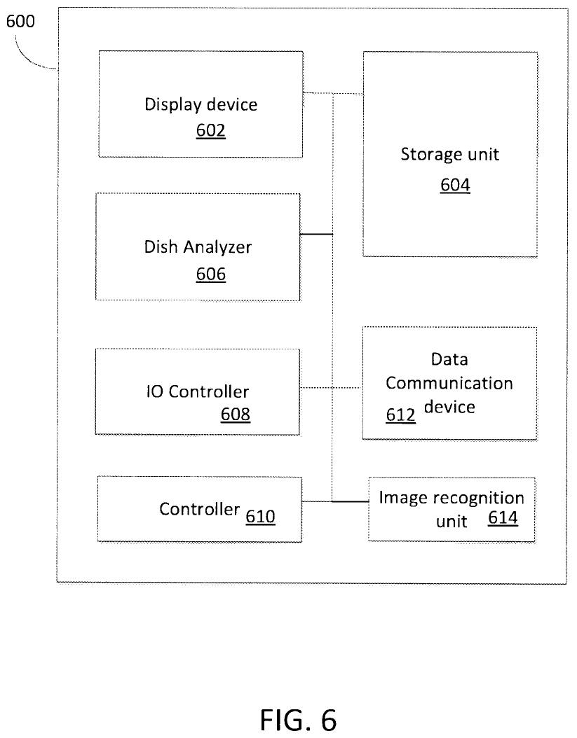

FIG. 6 is a schematic 600 for a special server 102 that implements the present invention. The server include a display device 602 for interfacing with the publisher 110 and a storage unit 604 for storing dish information, customer information, restaurant information, and the program code (computer program) for the server. The server includes also a data communication device 612, an IO controller 608, a controller 610, a dish analyzer 606, and an image recognition unit 614. The data communication device 612 is for interfacing with remote customers and restaurants and the IO controller 608 controls input/output for the server. The controller 610 executes the program code stored in the storage unit 604. The image recognition unit 614 helps to identify a dish when a consumer requests a dish based on a photo instead of providing the dish name or description. The dish analyzer 606 can be internal component that performs chemical analysis on the actual food; the dish analyzer 606 may also be an external device connected to the server. The chemical analysis may provide basic information about the percentage of sodium, sugar, and fat on the actual food. Alternatively, the dish information stored in the storage unit 604 may be received from outside sources such as food analysis laboratory and the dish information may list amount of calories, protein, and other information commonly listed on the food labels.

The DMAP system of the present invention improves the food ordering service by making easier for the consumers to enter their desired dish information and also assist the consumers to make a selection if the consumers do not have a clear idea about what they want to eat. For example, when a consumer receives a photo of a dish that his friend had and praised a lot, the consumer may want to order the same dish even he does not know the actual name of the dish. He can upload the photo to the DMAP system and the photo is received by the server through the data communication device 612. The photo is sent to the image recognition unit 614 for analysis. The photo may be recognized as a photo for dish XXX and this information is sent back to the consumer along with any other information related to dish XXX, such as whether the dish XXX is spicy, salty, or sweet. The consumer can confirm his acceptance and finish the ordering process.

In another situation, for example, the consumer wants to try some new dish but is not sure what meets his appetite. The DMAP system will prompt for the consumer's preference, whether he wants some hot and spicy flavor or he wants something light, not salty and greasy. If the consumer indicates he wants Mexican food with some vegetable, not too salty, the DMAP system may provide a list of Mexican food that fits these criteria.

The DMAP system also helps the publisher 110 or platform owners to improve their service to their customers. The DMAP system allows a publisher to improve the description of each dish on their dish catalog. The DMAP system allows the publisher to create a dish catalog or menu, not only by entering the traditional names, but also by the description of the flavor, taste, appearance, and nutritional information. When compiling the dish catalog, the publisher 110 may start with dish menus commercially or available on the Internet and may enhance the dish information by sending the dish to a chemical lab for nutritional analysis, alternatively the publisher may perform a simple analysis of sodium content through a small device connected to the server.

When in use, the DMAP system will take an order from a consumer either by taking input directly from the consumer or by asking the consumer's preference, then make suggestions. The consumer's order is recorded in a consumer database and this consumer database records the consumer's preference. The consumer database is used when the consumer places the order next time. The DMAP system either suggest some dish that may match the consumer's taste or suggest something that the consumer has not tried before. The DMAP system will take the order and also the payment from the consumer before forwarding the consumer's selection to a selected restaurant.

After the consumer's order is taken, the DMAP system checks the restaurant database for a suitable restaurant. The selection is based primarily on the restaurant's ability to fulfill the order and also based on the past consumer feedback. The selection criteria may include preference for those restaurants that have a special agreement with the publisher. After the selection, the order is sent to the selected restaurant for fulfillment. An estimate delivery time is sent to the consumer and a follow up email is also sent to the consumer to survey the satisfaction of the order.

While the invention has been particularly shown and described with reference to a preferred embodiment thereof, it will be understood by those skilled in the art that various changes in form and detail may be made without departing from the spirit and scope of the present invention as set forth in the following claims. Furthermore, although elements of the invention may be described or claimed in the singular, the plural is contemplated unless limitation to the singular is explicitly stated. It is foreseeable that different features described in different passages may be combined.

1. A method for assembling a dish database comprising:
receiving a description for a food item;
performing chemical analysis on a sample for the food item;
receiving a result from the chemical analysis; and
populating the dish database with the result and the description.

2. The method of claim 1, further comprising:
receiving an image for the food item;
receiving a list of ingredients for the food item; and
populating the dish database with the image and the list of ingredients.

3. The method of claim 1, further comprising:
receiving palate information for the food item;
populating the dish database with the palate information.

4. A method for receiving an order from a consumer comprising:
displaying a menu to the consumer;
receiving an input from the consumer;
if the input is an audio input, performing a voice recognition on the audio input to obtain a name for a food item;
if the input is an image input, performing an image recognition on the image input to obtain a name for the food item;
if the input is a list of ingredients, performing a search on an ingredient database for the list of ingredients to obtain a name for the food item;
checking the name against a dish database to see if the food item is available;
if the food item is not available, generating a palate index for the food item;
if the food item is not available, selecting a substitute food item based on the palate index; and
if the food item is not available, presenting the substitute food item to the consumer.

5. The method of claim 4, wherein the palate index is generated by checking savoriness, temperature, sweetness, satiety, crispiness, and liquidity of the food item.

6. The method of claim 4, further comprising:
if the food item is accepted by the consumer, selecting a restaurant for processing the order for the food item according to predefined criteria;
updating a consumer database with the food item; and
updating the dish database with the food item.

7. The method of claim 6, wherein the predefined criteria include geographical information and special agreement with the restaurant.

8. The method of claim 6, further comprising, if the order for the food item is not accepted by the restaurant, sending the order to another restaurant.

9. An apparatus, for receiving an order from a consumer, comprising:
a display device for displaying a menu to a consumer;
a data communication device for interfacing with remote consumers and restaurants;
an IO controller for controlling input/output for the apparatus;
a storage unit for storing dish information, customer information, restaurant information, and program code;
a dish analyzer for analyzing content of food item; and
a controller for executing the computer programs that causes the apparatus to:
displaying a menu to the consumer;
receiving an input from the consumer;
if the input is an audio input, performing a voice recognition on the audio input to obtain a name for a food item;
if the input is an image input, performing an image recognition on the image input to obtain a name for the food item;
if the input is a list of ingredients, performing a search on an ingredient database for the list of ingredients to obtain a name for the food item;
checking the name against a dish database to see if the food item is available;
if the food item is not available, generating a palate index for the food item;
if the food item is not available, selecting a substitute food item based on the palate index; and
if the food item is not available, presenting the substitute food item to the consumer.

10. The apparatus of claim 9, wherein the controller executing the computer programs that causes the apparatus to:
if the food item is accepted by the consumer, selecting a restaurant for processing the order for the food item according to predefined criteria;
updating a consumer database with the food item; and
updating the dish database with the food item.

11. The apparatus of claim 10, wherein the controller executing the computer programs that causes the apparatus to, if the order for the food item is not accepted by the restaurant, sending the order to another restaurant.

12. The method of claim 11, wherein the predefined criteria include geographical information and special agreement with the restaurant.

13. The method of claim 11, wherein the palate index is generated by checking savoriness, temperature, sweetness, satiety, crispiness, and liquidity of the food item.
